(19) United States
(12) Patent Application Publication
(10) Pub. No.: US 2017/0112587 A1
(43) Pub. Date: Apr. 27, 2017

(54) Protective Shield and Handle for Surgical Lamps

(71) Applicant: Jonathan R. Weiser, Fort Lauderdale, FL (US)

(72) Inventor: Jonathan R. Weiser, Fort Lauderdale, FL (US)

(21) Appl. No.: 15/299,821

(22) Filed: Oct. 21, 2016

Related U.S. Application Data
(60) Provisional application No. 62/244,249, filed on Oct. 21, 2015.

(51) Int. Cl. A61B 90/30; F21V 21/40

(52) U.S. Cl. CPC A61B 90/30; F21V 21/403; A61B 2090/308; F21W 2131/205

(57) ABSTRACT

A handle configuration for mounting to a lighthead, having a handle grip having a longitudinal axis defining a longitudinal direction of the handle grip, the handle grip having a base adjacent the lighthead in a mounted position of the handle configuration and the handle grip having a terminal end opposite the base, a wall extending from the mounting base in the longitudinal direction, the wall defining a gap between an inner surface of the wall and the handle grip for allowing a user's hand to grasp the handle grip and adjust a position of the lighthead.

### CROSS REFERENCE TO RELATED APPLICATIONS

This application claims benefit of U.S. Provisional Patent Application No. 62/244,249 filed Oct. 21, 2015, entitled Protective Shield and Handle for Surgical, the disclosure of which is incorporated herein by reference in its entirety.

### BACKGROUND OF THE INVENTION

a) Field of the Invention:

The present invention is in the field of surgical illumination and sterilization related to the large overhead light fixture used to illuminate a surgical site or other site of interest in a medical procedure, whether in an operating room or any other area where sterilization is critical. The surgical light fixture, typically includes a plurality of lights, the fixture being mounted to the ceiling, a wall or may be on a pedestal. Though the term large is used, surgical lights come in varying sizes and diameters.

A surgical light fixture, also referred to as an operating light or surgical lighthead, is a medical device intended to assist medical personnel during a surgical procedure by illuminating a local area or cavity of a patient. A combination of several surgical light fixtures is often referred to as a surgical light system.

In use, the lighthead is typically manipulated by the surgeon, during a sterile procedure on a patient. In the procedure, the surgeon and other medical personal will wear sterile gloves on their hands. The surgeon's hand will hold the handle of the lighthead to move and direct the beams of light from the lighthead to the area desired to be illuminated by the surgeon during the procedure. A surgeon or other medical person may adjust the lighthead several times during a procedure, each time by holding the handle of the lighthead. Though the lighthead fixture holding the lights is not sterile, it is desirable that the light handle which is a part of the lighthead and used by the surgeon to adjust the lighthead, and which comes in contact with the surgeon's sterile gloved hand, be sterile. It is known in the art to use replaceable lighthead handles or sterile covers for the lighthead handle, “light handle covers” on the lighthead handle to create an area on the lighthead handle “light handle” that is sterile.

These replaceable sterile handles or covers, as known in the art, can either slide onto the lighthead handle, for example where the lighthead has a male type handle connector. Alternatively a lighthead can have a female type handle connector in the form of an internal threaded opening to receive a replaceable handle with a male end to screw into the female connector of the lighthead.

Typically the replaceable handle covers are replaced at least for every procedure, and often times, they are necessarily replaced during the procedure, when the replaceable sterile handle cover comes in contact with an object that is not sterile. The surgeon's sterile gloved hands are sterile, and it is this sterile glove on the surgeon's hand that contacts the light handle when the light handle is adjusted or manipulated. However during a procedure other objects may accidentally come in contact with the light handle which contaminates the sterile handle cover. Such objects may be a person's head, a surgeon's head or nurse's head or an attendant's head, whether or not the head is covered, or for example an adjacent lamp or lighthead that is not sterile can contact the light handle and contaminate the light handle. Thus, during a procedure if it is noticed that a nonsterile object contacts the sterile light handle cover then the now contaminated light handle cover must immediately be removed and replaced with a clean sterile light handle cover. This replacement is typically performed by a person in the operating room other than the surgeon. This replacement takes time and interrupts the medical procedure while the contaminated light handle cover is removed, disposed and then replaced with a new sterile light handle cover. During a procedure, it is possible that this contact goes un-noticed. Most importantly, this contact between the adjacent objects and the light handle causes bacterial contamination of the surgical field and that greatly increases the risk of infection that is caused from the contaminated light handle.

### SUMMARY OF THE INVENTION

The present invention is a shield or guard and light handle to be used on the handle of a lighthead in an operating room during a surgical procedure, when it is required that the light handle of the lighthead be sterile. The shield of the present invention prevents or limits other objects from coming in contact with the sterile light handle cover of the lighthead that the surgeon's sterile gloved hand contacts when the surgeon is manipulating the lighthead. The present invention can also provide tactile feedback to alert the person touching the light handle, when the sterile handle is contacted by the person.

The shield of the present invention can take several forms, a shade that extends around the light handle allowing sufficient room for a surgeon's sterile gloved hand to reach in to grab or hold on to the sterile light handle. The shade may be a solid wall surrounding the light handle, or it may have two or more elongated strut members or wall portions surrounding the sterile light handle cover, such that the wall prevents an object from coming in contact with and contaminating the sterile light handle cover.

Further the shield can also be at the base or terminal end of the light handle cover to likewise maintain the sterility or integrity of the sterile handle and/or limit contamination from an object coming in contact with the protected grip portion of the sterile light handle.

Additionally, the shield can extend below the plane of the terminal end of the light handle to prevent objects from coming in contact with the protected grip portion of the sterile light handle. These embodiments can be combined as well.

Therefore, it is a primary object of the present invention to protect the sterile light handle and limit contamination of the sterile light handle preventing contact from non-sterile objects. Thus diminishing the risk of infection.

It is another object of the present invention to guard against the unknown of not knowing the sterile handle cover came in contact with a non-sterile object by providing tactile feedback when contact is made.

It is a further object of the present invention to maintain the sterility of the grip portion of the sterile light handle and limit contact, whether known or not, with contaminated objects.

It is another object of the invention to place guards or obstructions around the sterile light handle. These obstructions include but are not limited to a wall, a shade, an elongated strut member, fingers or a base guard or flange at the terminal end of the light handle or the light handle cover.

It is a further object of the invention to increase the potential for recognizing contact with the light handle by creating notice or feedback or tactile feedback of the contact to the person causing the unwanted contact, by having a shield to protect the sterile area of the handle. This notice or feedback can be by an audible or visual notice or feedback, for example an audible alarm could be triggered or a light could be switched on to provide notice or feedback in the operating room to not only alert the person that caused the unwanted contact but to alert the person in charge of replacing the sterile light handle to replace the contaminated light handle.

It is another object of the invention that the shield of the present invention limit contact with the sterilized area of the light handle. Such that the shield will create feedback to give notice, a tactile feedback, to the person causing the contamination, so the contaminated handle can be replaced quickly. By way of example the tactile feedback can be any form of tactile communication to the person causing the contamination to provide notice of the contamination or possible contamination of the light handle or lighthead itself.

It is a further object of the invention to protect the sterile light handle from lateral contact at the sides of the handle.

It is an object of the present invention to prevent contamination of the light handle cover and thus limiting changing of the sterile handle cover.

It is another object of the invention to have a protective shield around the sterile light handle cover at a distance that is sufficient enough to accommodate insertion and withdrawal of a user's or surgeon's hand to hold the sterile area of the light handle.

It is another object of the invention to maximize surgery time and prevent interruptions during surgery due to contamination of the sterile light handle by limiting contact of contaminated objects with the sterile portions of the light handle and cover.

It is another object of the present invention that the shield be made of a material that is disposable, alternatively the shield can be reusable after use by treatment with a sterilizing machine as known in the art.

It is another object of the invention that the shield will not interfere with any opening in bottom of the handle cover that is used for accessories such as a camera lens or other device.

It is also an object of the invention that the shield includes a light handle cover portion, alternatively the shield can attach to lighthead handle while a separate or independent light handle cover is used. The sterile light handle cover may be independent of the shield of the present invention.

It is another object of the present invention that guard members or struts or portions of a wall can depend from the base of the lighthead or the peripheral edges of a lighthead to prevent an adjacent lighthead from coming in lateral contact with the handle of the lighthead.

In an alternate embodiment, the shield is incorporated into the lighthead with the handle therein.

It is accordingly an object of the invention to provide a handle configuration for mounting to a lighthead, the handle configuration having a handle grip having a longitudinal axis defining a longitudinal direction of the handle grip, the handle grip having a base adjacent the lighthead in a mounted position of the handle configuration, the handle grip having a terminal end opposite the base, a wall extending from the mounting base in the longitudinal direction, the wall defining a gap between an inner surface of the wall and the handle grip for allowing a user's hand to grasp the handle grip and adjust a position of the lighthead.

With the foregoing and other objects in view, with the handle configuration the wall has a length, the length has an extent in a radial direction of the handle grip to achieve the gap.

In accordance with another feature of the invention, the wall is a continuous wall that is continuous in a circumferential direction around the handle grip.

In accordance with an added feature of the invention, the wall is a plurality of spaced apart walls distributed about a circumference of the base.

In accordance with an additional feature of the invention the plurality of spaced apart walls are spaced apart from one another along the circumference at a distance sufficient for allowing the user's hand to pass between adjacent spaced apart walls and into the gap.

In accordance with yet an additional feature of the invention, the handle grip has a hole formed therein that extends in the longitudinal direction, the hole being dimensioned for receiving a handle of the lighthead.

In accordance with yet another added feature of the invention the terminal end has a flange for shielding a gripping surface of the handle grip.

In accordance with still another added feature of the invention, the gripping surface has a diameter between one and one half to four and one half centimeters and the flange has a diameter between three and one-half to seven and one-half centimeters.

In accordance with still another added feature of the invention, the inner surface of the wall is spaced seven to eight centimeters from an edge of the flange.

10. The handle configuration according to claim 6 wherein said hole is dimensioned to have a friction fit with the handle.

11. The handle configuration according to claim 7, wherein an inner surface of said wall is spaced from said gripping surface by six to fourteen centimeters.

In accordance with still another added feature of the invention, the base end has a threaded stud for mounting the handle configuration to a female thread formed in the lighthead.

In accordance with still another added feature of the invention, the wall extends in the longitudinal direction substantially as far as the terminal end is spaced from the base.

In accordance with still another added feature of the invention, the handle configuration triggers an audible or visual notice with an audible alarm or switches on a light to provide notice or feedback of any contact with the handle grip.

In accordance with still another added feature of the handle shield for being mounted to a lighthead and shielding a handle of the lighthead, the shield has a mounting base having an opening formed therein dimensioned for receiving the handle therein and allowing the mounting base to be slid onto the handle, the mounting base defining a longitudinal direction of the shield along a longitudinal axis of the handle and defining a radial direction relative to the longitudinal direction, a wall extending from the mounting base in the longitudinal direction, the wall defining a gap between an inner surface of the wall and the handle for allowing a user's hand to grasp the handle and adjust a position of the lighthead.

### BRIEF DESCRIPTION OF THE DRAWINGS

FIG. 1A is a perspective view of the shield of the present invention showing a light handle cover with a shield having four elongated wall members extending from a base of the light handle cover and an enlarged terminal end portion to limit contact of the grip portion of the handle cover with non-sterile objects.

FIG. 1B is a side view of the shield of the present invention shown in FIG. 1A.

FIG. 1C is a bottom view of the shield of the present invention shown in FIG. 1A.

FIG. 1D is a sectional view of a wall portion taken along line B-B in FIG. 1B.

FIG. 2A is a side view of an alternate embodiment where the handle portion, the light handle, of the shield does not have a flange at the terminal end.

FIG. 2B is a side view of an alternate embodiment where the shield portion of the light handle cover is at the terminal end of the light handle formed by a flange portion of the light handle and there is no upper shield portion.

FIG. 2C is an alternate embodiment where the upper shield portion is independent of and without a handle cover portion.

FIG. 2D shows a light handle having a male handle connector constructed and arranged for engagement to a lighthead having a threaded female opening.

FIG. 3 is a perspective view of an alternate embodiment where the shield portion is a shade or continuous wall that surrounds the light handle cover.

FIG. 4 is a top view of the embodiment shown in FIG. 3.

FIG. 5 is a perspective view of a surgical light system mounted to a wall such as a ceiling wall and supporting two lightheads. Each lighthead having a handle shield system of the present invention as shown in FIG. 1 mounted on the lighthead handle of the lighthead.

FIG. 6 is a perspective view of the surgical light system shown in FIG. 5 further showing the shield of the present invention protecting the sterile light handle cover and preventing the non-sterile edge of the adjacent lighthead from contacting and contaminating the sterile lighthead handle cover.

FIG. 6A is a cross section of the shield of the present invention along lines A-A in FIG. 6 showing the shield of the present invention mounted on the handle of the lighthead system.

FIG. 7 is a perspective view of a surgical light system showing an alternate embodiment of the present invention, with the shield including outer walls attached to the lighthead as a part of the lighthead assembly.

FIG. 8 is a perspective view of a surgical light system showing an alternate embodiment of the present invention where the shield and light handle are within a portion of the light fixture and the terminal end of the light handle extends to be approximately flush with the lower edge of the lighthead fixture.

FIG. 9A is a partial cross sectional view of the surgical light system of FIG. 8 showing the male handle connector but without the shield and light handle

FIG. 9B is a partial cross sectional view of the surgical light system of FIG. 8 showing the shield and light handle.

FIG. 10 is an example of a prior art lighthead system showing a person holding the light handle and showing the head of a person in close proximity to the lighthead with the possibility of the head of the person contacting the light handle and resulting in the contamination of the light handle.

FIG. 11 is a side view of a light assembly showing the male handle connector.

FIG. 12 is a partial cross section view of FIG. 11, showing the male handle connector.

FIG. 13 is a partial cross sectional view of a light head system where the light head system has a female connector to receive a light handle.

FIG. 14 is a side view of a shield with handle showing a male connector to mate with the female connector of FIG. 13.

### DESCRIPTION OF THE PREFERRED EMBODIMENTS

The protective handle shield 10 of the present invention, as seen in the figures includes a handle grip or light handle 20. Light handle 20 has a terminal end 22 and shield 10 has a base 30. Base 30 has an upper flange 32 having a circumferential wall 34. Depending from the base 30 are one or more walls 36. The light handle 20 defines a longitudinal axis LA. The wall 36 extends from base 30 in the longitudinal direction of the longitudinal axis LA. Additionally, wall 36 may also extend in a radial direction from light handle 20 along longitudinal axis LA.

Light handle 20 has a center opening 12 for rigidly mounting shield 10 on to shield 10. As is known in the art, the internal surface of said opening 12 may have a plurality of elongated ribs 57b, to increase friction against male handle connector 57. The shield 10 has a mounting interface that in a first embodiment is for mounting shield 10 to a male connector 57 of a lighthead 50. In a second embodiment, the mounting interface is a threaded female opening 58 to receive the male handle connector 59. Irrespective of the mounting interface between shield 10 and lighthead 50, there is a light handle 20 as shown which has a cylindrical shape, though light handle 20 can be of any shape, including oval, square, rectangular or octagonal. Light handle 20 has an outer wall 21 and an inner wall 23 which would be the inner wall of opening 12.

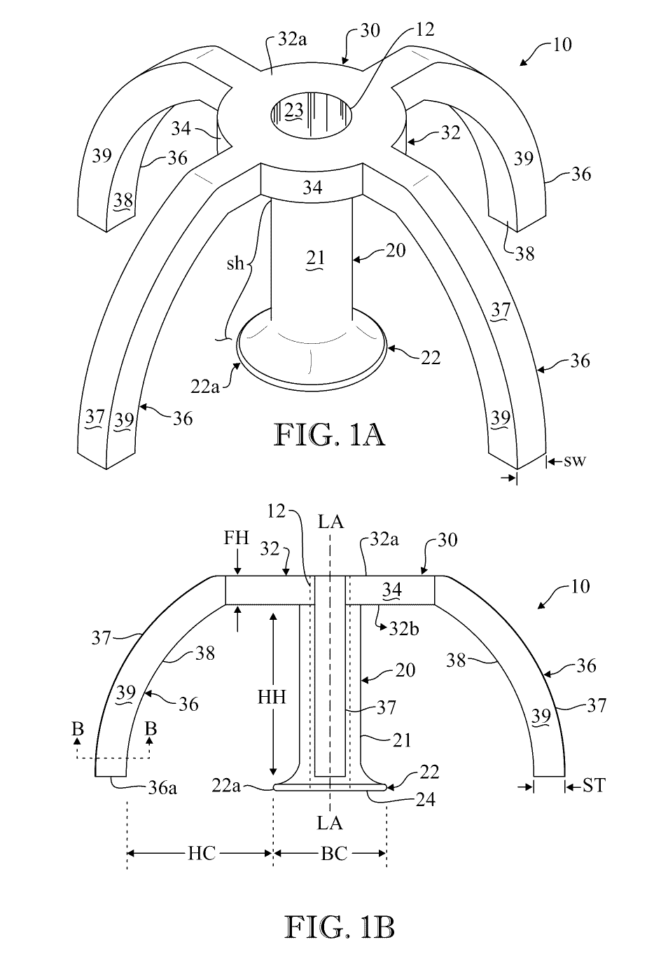

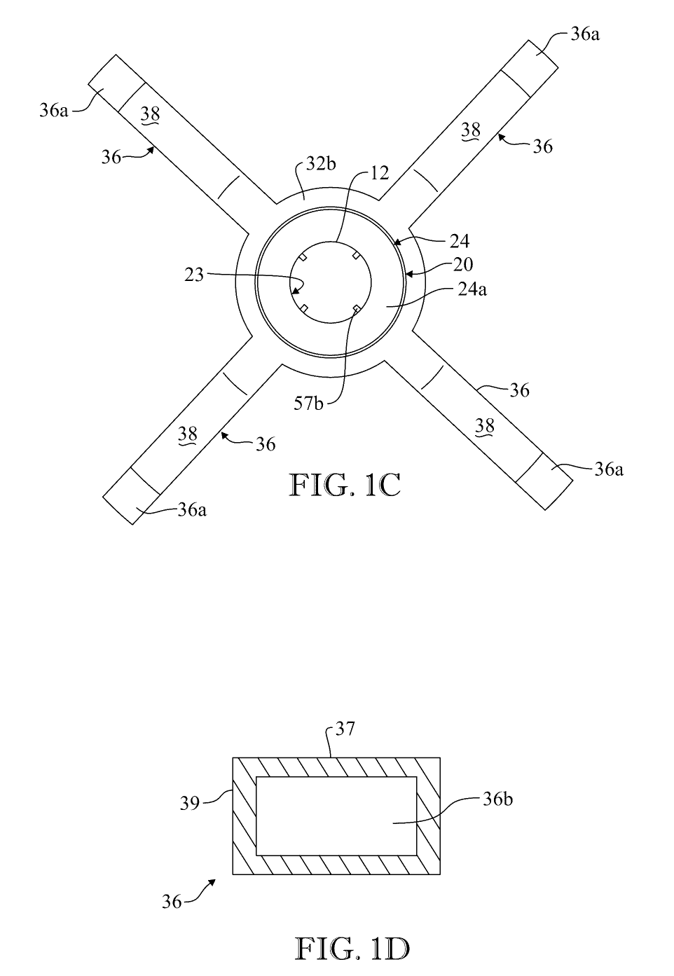

Base 30 has an upper flange 32 which is circular, but can be of any shape, including oval, square, rectangular or octagonal. Upper flange 32 has a lighthead side 32a, a handle terminal side 32b and a flange height FH, which is also the thickness of circumferential wall 34. Walls 36 have an outer surface 37, an inner surface 38 and side walls 39. Walls 36 have a wall thickness ST substantially the same as the flange height FH of circumferential wall 34, and a wall width SW. The wall thickness ST is substantially the same as the wall width SW. The wall thickness will depend upon the material that wall 36 is made. The stronger the material, the smaller the thickness. If metal, in a preferred embodiment the wall width SW could be one centimeter and the wall thickness ST could be one tenth of one centimeter. If made of plastic SW could be one centimeter and ST could be one centimeter. The walls 36 can be solid or hollow having a cross section B-B taken from FIG. 1B as shown in FIG. 1D. Walls 36 are sufficiently strong to bend, but not break when wall 36 comes in contact with force from an external object.

Light head 20 has a sterile handle cover grip portion SH defined by height HH along the outer surface 21 of light handle 20. This grip portion SH or sterile handle cover portion SH comes in contact with the surgeon's sterile gloved hand, and this grip portion SH is preferably maintained in a sterile state by the shield 10 of the present invention that limits contamination of the grip portion SH along light handle 20

As seen in FIG. 1C, the terminal end 24 of light handle 20 has a bottom surface 24a. This bottom surface 24a can cover the entire bottom portion of light handle 20, such that center opening 12 is a blind hole, or as shown in FIG. 1C the bottom surface 24a is an annular ring with a center opening 12 to receive the lighthead male handle connector 57 from the light head 50. Center opening 12 is constructed and arranged to match the diameter of connector 57 and is dimensioned so that shield 10 will fit on connector 57 with a friction fit. Alternatively a thumb screw (not shown) can be used to rigidly hold shield 10 on connector 57.

Where the male handle connector 57 from the lighthead 50 includes auxiliary equipment, such as a camera, this opening 12 provides the auxiliary equipment freedom to operate. The foot 36a of each wall 36 may be solid with no opening or there may be an opening in the foot 36a, conforming to the sectional view with opening 36b shown in FIG. 1D.

The terminal end 22 has a flange 22a for shielding the grip portion SH. Flange 22a has a diameter BC which is greater than the diameter of light handle 20. The terminal end 22 thus provides protection of the grip portion SH of handle cover 20. It is this terminal end 22 with flange 22a that protects the grip portion SH even if an object comes in contact with the terminal end 22 of light handle 20. Additionally, terminal end 22 and bottom surface 24a of light handle 20 and flange 22a provide tactile feedback to anyone touching or coming in contact with the shield 10. As seen in FIG. 1B a hand clearance distance HC exists between the peripheral edge of flange 22a and the inside surface 38 of wall 36. In a preferred embodiment the distance HC could be 7 to 8 centimeters. The distance BC, the diameter of flange 22a could be five and one half centimeters and the diameter of grip portion SH could be 3 centimeters. However, in situations where a camera or other instrument is used within handle connector 57, the diameter of grip portion could be 6 to 12 centimeters to accommodate the camera or other instrument.

As heretofore mentioned, it is possible to increase the potential for recognizing an inadvertent contact with the light head 20 by creating notice or feedback of the contact to the person that causing the unwanted contact or to others in the vicinity of the shield 50. This notice or feedback can be by an audible or visual notice or feedback, for example an audible alarm could sound, or a light could flash to provide notice or feedback in the operating room to not only alert the person that caused the unwanted contact but to alert the person in charge of replacing the sterile light handle to replace the contaminated light handle. Such audible or visual notice can be constructed and arranged as is known in the art.

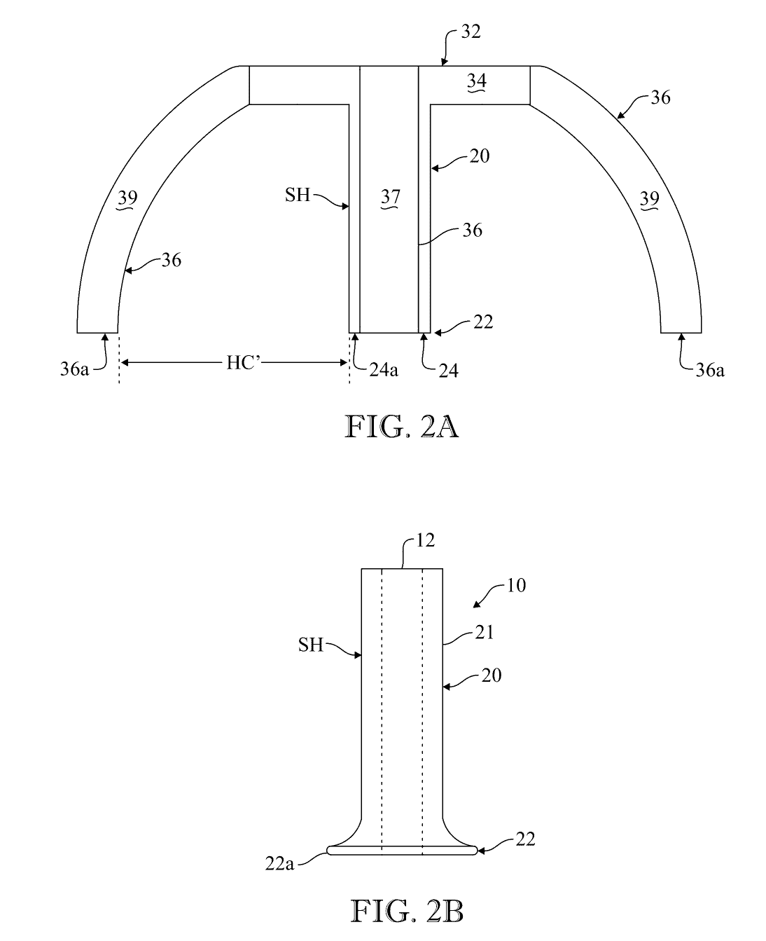

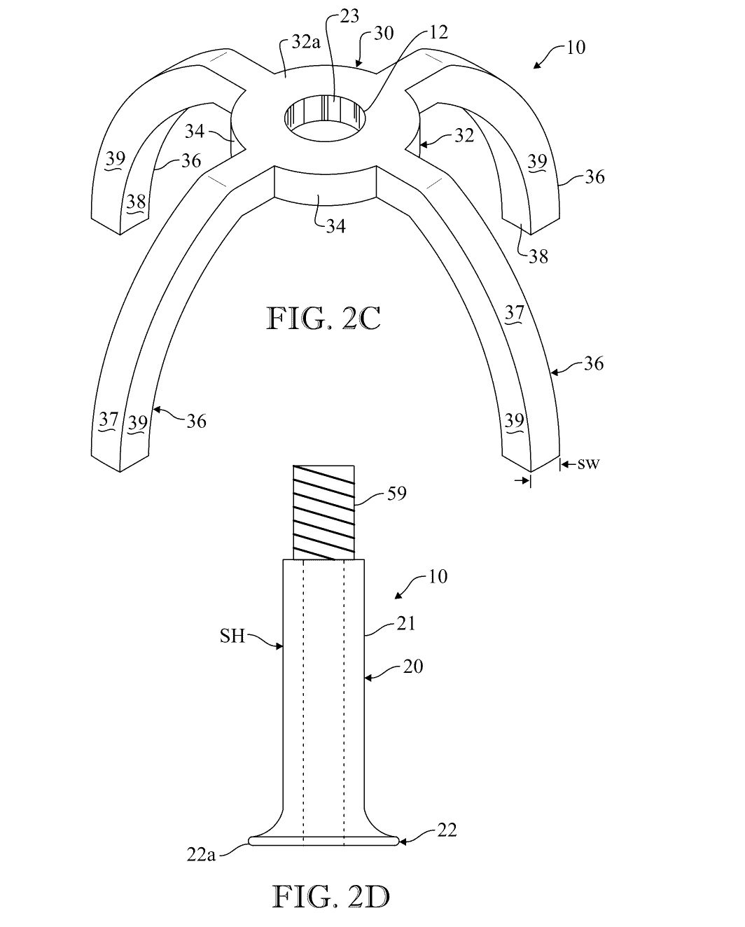

An alternate embodiment of the shield of the present invention is shown in FIG. 2A where the light shield 20 does not have a flange 22a. In this embodiment shown in FIG. 2A, the hand clearance HC′ is increased as compared to hand clearance HC shown in FIG. 1B, because of the elimination of the flange 22a light handle 20. Notwithstanding the terminal end 22 of light handle 20 in this embodiment can still provide tactile feedback.

In another alternate embodiment, shown in FIG. 2B the light handle 20 includes flange 22a, but does not have walls 36. This offers the benefit of a light handle 20 with the flange 22a, limiting contamination of the grip portion SH of light handle 20 without a wall 36. The terminal end 22 of light handle 20 in this embodiment can still provide tactile feedback.

An additional embodiment is shown in FIG. 2C where the base 30 does not include a light handle 20. In this embodiment, the upper base 32 and accompanying wall 36 can be used independent of the type of handle cover used on male connector 57. In such an instance, this handle shield system 10 embodiment shown in FIG. 2c would be mounted on a lighthead 50 by sliding the male connector 57 through opening 12. In this manner the male connector 57 would remain uncovered. Then a light handle cover such as that shown in FIG. 2B or any other generic light handle cover known in the art, could be mounted on the male connector 57.

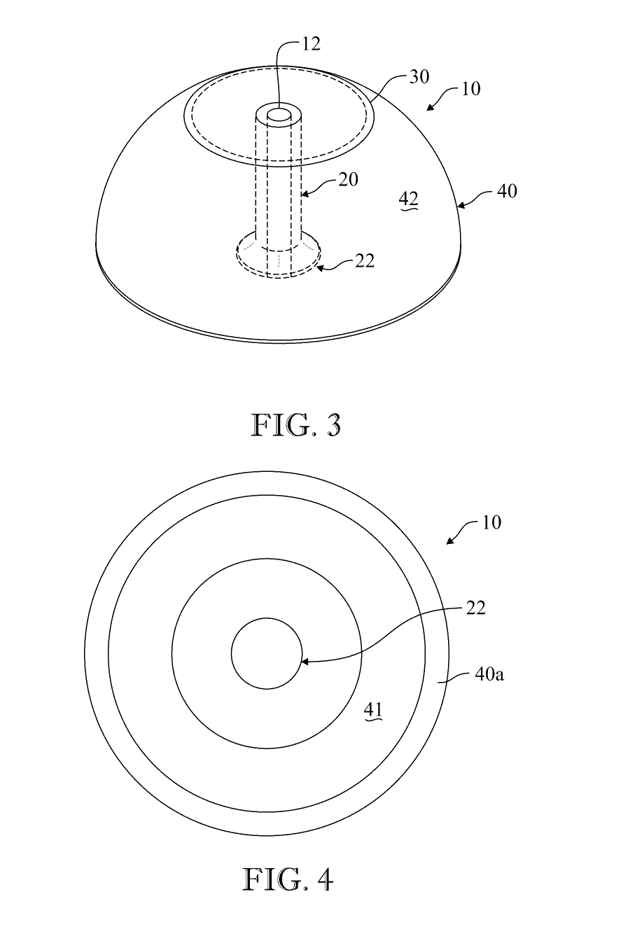

Another alternate embodiment of the shield of the present invention is shown if FIGS. 3 and 4, where the shield 10 is a continuous wall 40 having a cross section substantially in the shape of wall 36. Wall 40 has a continuous foot 40a and an inner surface 41 and an outer surface 42. Wall 40 allows sufficient hand clearance HC for the surgeon or other user to insert the sterile gloved hand within shield 10 between wall 40 and light handle 20. Wall 40 protects the grip portion SH of light handle 20 limiting contact from contaminated objects. Terminal end 22 limits contact with the grip portion SH and provides tactile feedback as well.

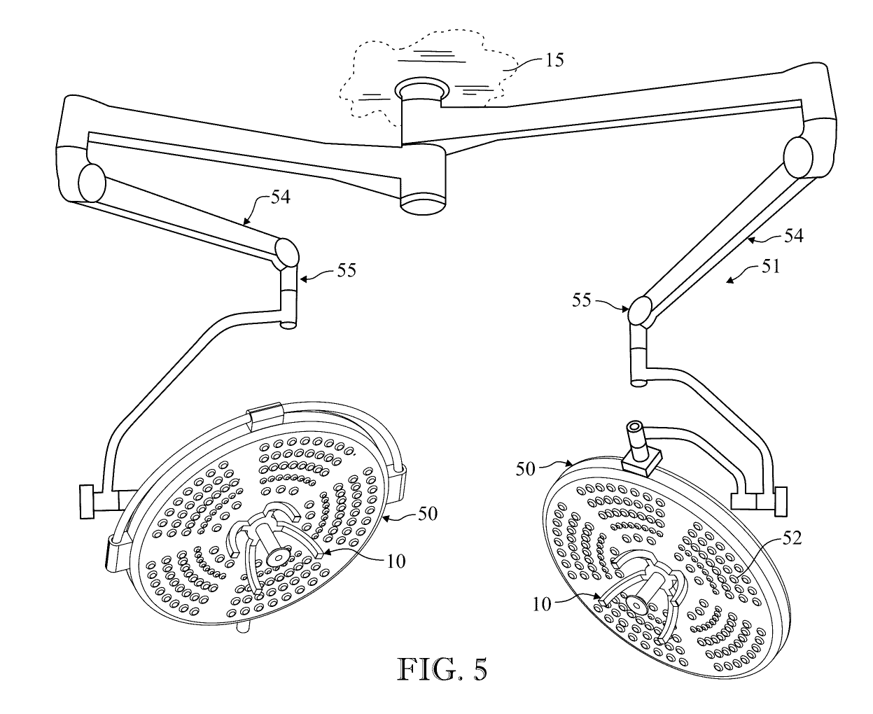

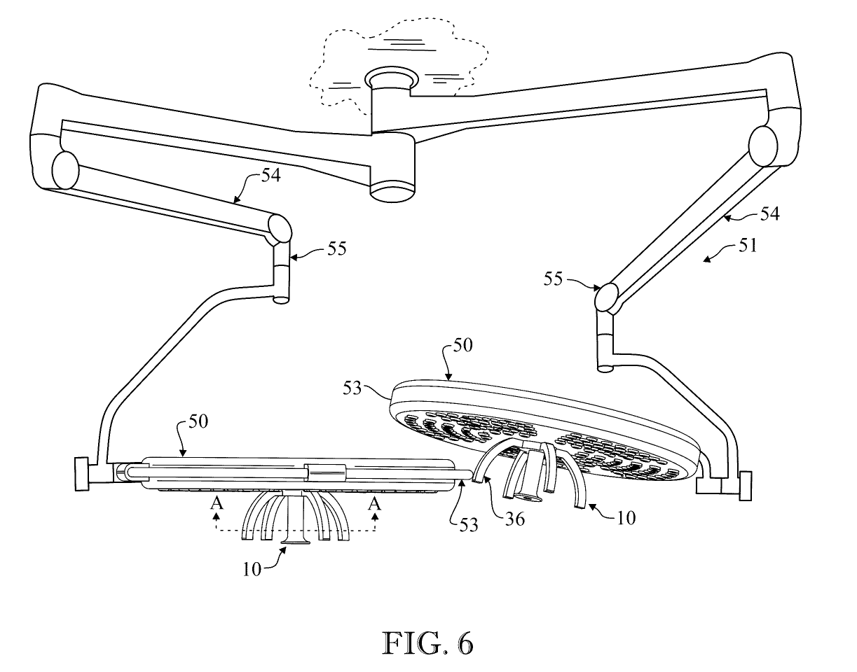

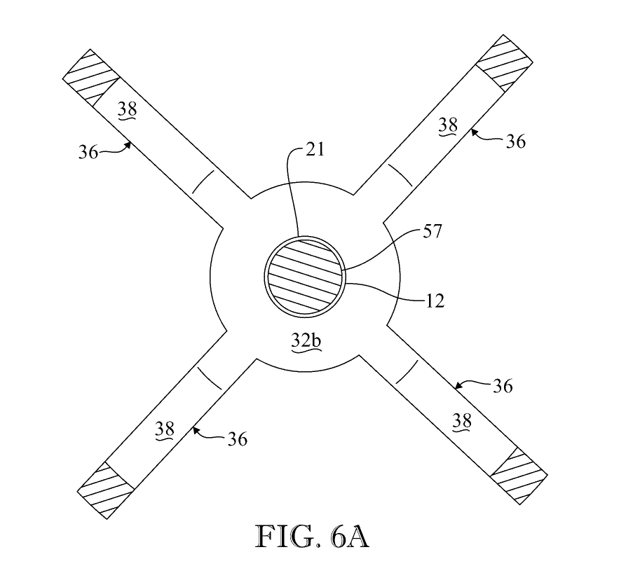

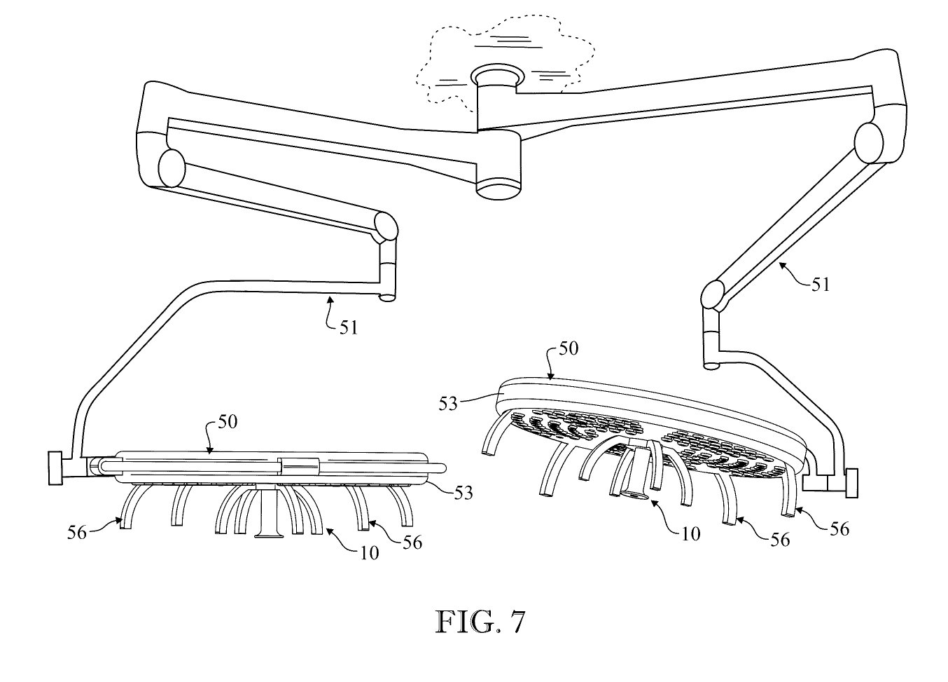

A lighthead system 51 with two lightheads 50 is shown in FIGS. 5, 6 and 7. Each lighthead 50 has installed thereon a handle shield system 10 of the present invention. Each lighthead 50 has a male handle connector 57 and a plurality of lights 52. These lights 52 may use LED bulbs. The male handle connector 57 of the lighthead 50 cannot be seen since the handle shield cover 10 of the present invention is mounted on the male connector 57. The male connector 57 is shown in cross section in FIG. 6A which is taken along lines A-A in FIG. 6. The lighthead systems 51 shown have the protective shield 10 of the present invention, and can use any of the alternate embodiments of the present invention disclosed herein.

Lighthead system 51 is mounted to a wall or ceiling 15 and includes two lightheads 50. Each lighthead 50 is suspended by a series of linked arms 54 with elbows and joints 55 that allow the lightheads 50 to be strategically moved and placed so the projected light from each lighthead 50 can be in a desired position.

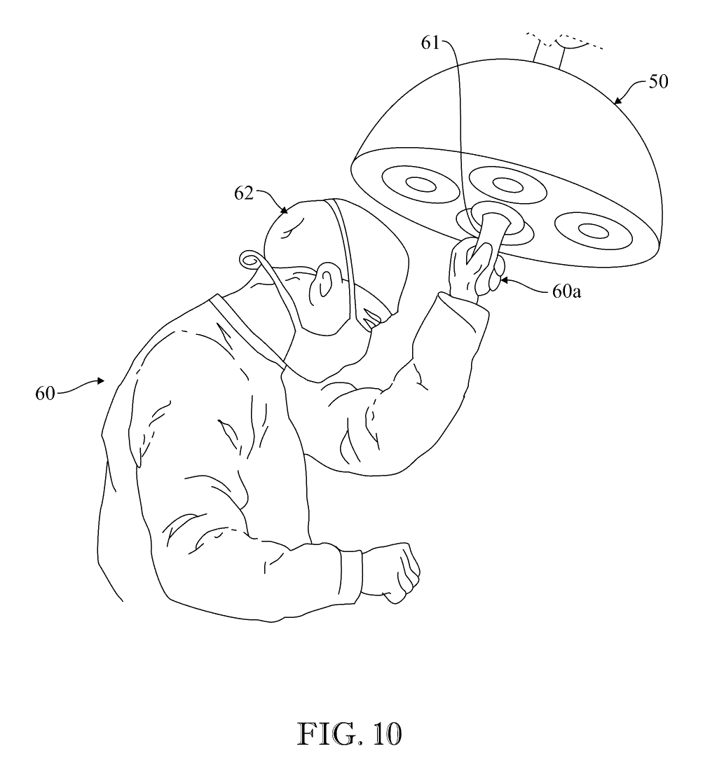

As seen in FIG. 6, in use, the two closely spaced lightheads 50 often come in close contact with one another and it is possible that a peripheral edge 53 of a lighthead 50 would contact the adjacent lighthead 50. Prior to the present invention, when this would happen the peripheral edge 53 of a first lighthead 50 could contact the light handle 20 of an adjacent or second light head 50. This non-sterile portion of the lighthead 50 would thus contaminate a sterile portion such as the grip portion SH of the second lighthead 50. As can be seen in FIG. 6, the shield 10 of the present invention prevents contamination of the sterile grip portion SH from objects, when the peripheral edge 53 of lighthead 50 comes close to an adjacent lighthead 50, the walls 36 protect the sterile light handle 20 and the sterile handle portion SH and thus limits contamination from an adjacent lighthead 50. The shield 10 of the present invention limits lateral contact of the adjacent objects including lighthead 50. Adjacent objects can also include a head of a person in the vicinity of the lighthead 50. As a further example, as shown in FIG. 10, in the prior art, a surgeon or other person 60 in the vicinity of light 50 would adjust lighthead 50 using the person's hand 60a to hold light handle 61 which light handle 61 is preferably sterile. It can be seen in this view, which is similar in an actual operating room setting that the surgeon's head 62 will come close to the sterile light handle 61. If and when such an object as the surgeon's head 62 comes in contact with the sterile handle 61, the surgeon's head 62, not being sterile, will contaminate the previously sterile light handle 61. Then the light handle 61, will then need to be and will be replaced during the procedure to prevent contamination.

An alternate embodiment also includes the addition of peripheral walls 56 mounted on the peripheral edges 53 of the lighthead 50 to further protect the entire lighthead 50 and the handle shield system 10 from contact from adjacent objects including adjacent lighthead 50.

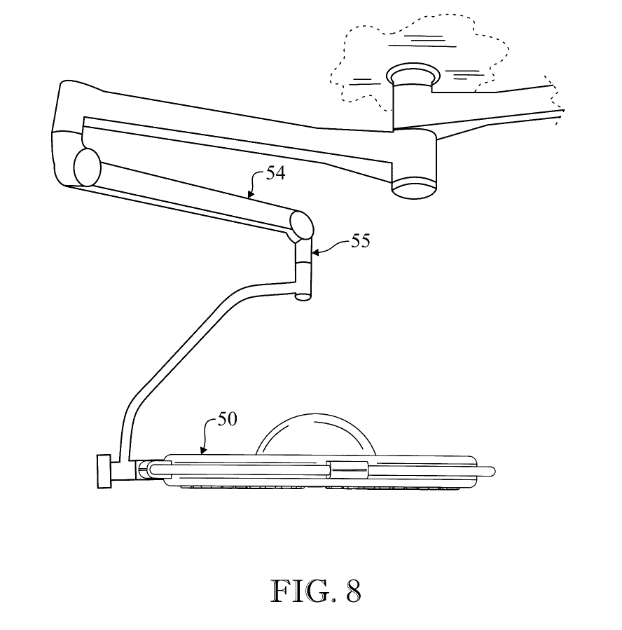

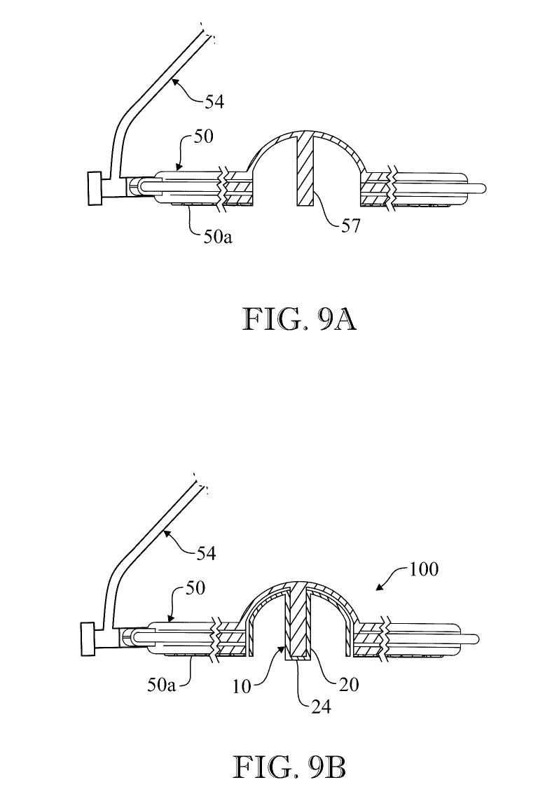

FIGS. 8, 9A and 9B disclose an alternate embodiment where the lighthead 50 incorporates the shape of the shield 10 and is constructed and arranged such that the terminal end 24 of light handle 20 is flush with the bottom surface 50a of the lighthead 50. In this embodiment the opening 12 of shield 10 would slide onto handle connector 57 as described hereinabove.

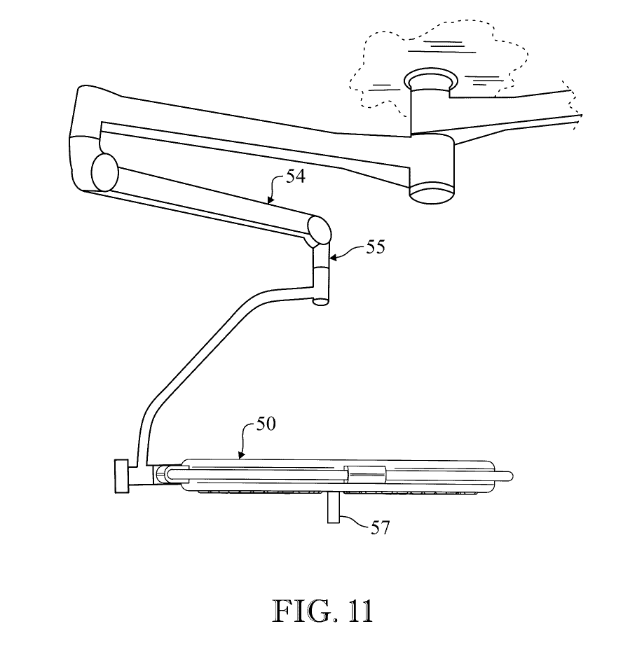

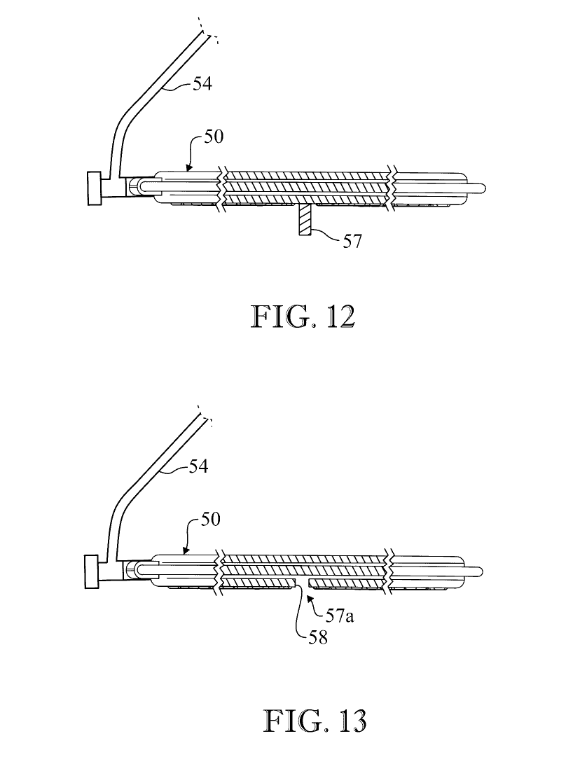

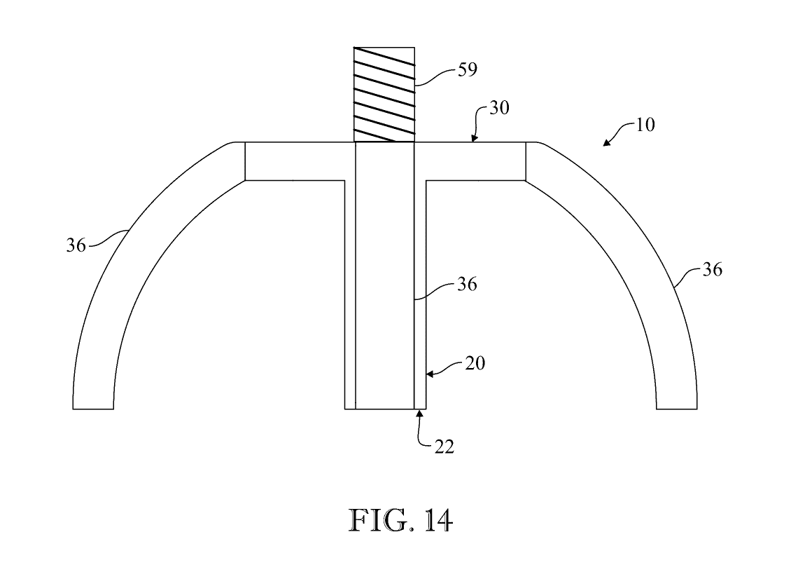

FIG. 11 shows a lighthead 50 with male handle connector 57. FIG. 12, shows a sectional view of a lighthead 50 of FIG. 11 with male handle connector 57. FIG. 13 shows an alternate design of a lighthead 50 that uses a female handle connector 57a where a threaded female opening 58 is constructed and arranged to receive a light handle as known in the art. For such designs, an alternate embodiment of shield 10 the present invention is shown in FIG. 14. In this embodiment, base 30 is constructed and arranged to have a mounting interface with light head 50 such that a male handle connector 59 will engage the threaded female opening 59 of lighthead 50 shown if FIG. 13. FIG. 2D shows an alternate embodiment of a shield 10 having a light handle 20 and a terminal end 22 having a male handle connector 59 constructed and arranged for engagement to a lighthead 50 having a threaded female opening 58.

While the invention has been described in its preferred embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A handle configuration for mounting to a lighthead, the handle configuration comprising:
a handle grip having a longitudinal axis defining a longitudinal direction of said handle grip, said handle grip having a base adjacent the lighthead in a mounted position of the handle configuration, said handle grip having a terminal end opposite said base;
a wall extending from said base in said longitudinal direction, said wall defining a gap between an inner surface of said wall and said handle grip for allowing a user's hand to grasp said handle grip and adjust a position of the lighthead.

2. The handle configuration according to claim 1, wherein said wall has a length, said length has an extent in a radial direction of said handle grip to achieve said gap.

3. The handle configuration according to claim 1, wherein said wall is a continuous wall that is continuous in a circumferential direction around said handle grip.

4. The handle configuration according to claim 1, wherein said wall is a plurality of spaced apart walls distributed about a circumference of said base.

5. The handle configuration according to claim 4, wherein said plurality of spaced apart walls are spaced apart from one another along the circumference at a distance sufficient for allowing the user's hand to pass between adjacent ones of said plurality of spaced apart walls and into said gap.

6. The handle configuration according to claim 1, wherein said handle grip has a hole formed therein that extends in the longitudinal direction, said hole being dimensioned for receiving a handle of the lighthead.

7. The handle configuration according to claim 6 wherein said hole is dimensioned to have a friction fit with the handle.

8. The handle configuration according to claim 1, wherein said terminal end has a flange for shielding a gripping surface of said handle grip.

9. The handle configuration according to claim 8 wherein said gripping surface has a diameter between one and one half to four and one half centimeters and said flange has a diameter between three and one-half to seven and one-half centimeters.

10. The handle configuration according to claim 9, wherein an inner surface of said wall is spaced 7 to 8 centimeters from an edge of said flange.

11. The handle configuration according to claim 8, wherein an inner surface of said wall is spaced from said gripping surface by six to fourteen centimeters.

12. The handle configuration according to claim 1, wherein said base has a threaded stud for mounting the handle configuration to a female thread formed in the lighthead.

13. The handle configuration according to claim 1, wherein said wall extends in said longitudinal direction substantially as far as said terminal end is spaced from said base.

14. The handle configuration according to claim 1, wherein said handle configuration triggers an audible or visual notice with an audible alarm or switches on a light to provide notice or feedback of any contact with said handle grip.

15. A handle shield for being mounted to a lighthead and shielding a handle of the lighthead, the shield comprising:
a mounting base having an opening formed therein dimensioned for receiving the handle therein and allowing the mounting base to be slid onto the handle, said mounting base defining a longitudinal direction of the shield along a longitudinal axis of the handle and defining a radial direction relative to said longitudinal direction;
a wall extending from said mounting base in said longitudinal direction, said wall defining a gap between an inner surface of said wall and the handle for allowing a user's hand to grasp the handle and adjust a position of the lighthead.
